(19) United States
(12) Patent Application Publication
(10) Pub. No.: US 2014/0180321 A1
(43) Pub. Date: Jun. 26, 2014

(54) DISCECTOMY DEVICES AND METHODS

(71) Applicant: Spine View, Inc., Fremont, CA (US)

(72) Inventors: Mark Dias, San Jose, CA (US); Al Mirel, Redwood City, CA (US); Robert May, Hayward, CA (US)

(73) Assignee: Spine View, Inc., Fremont, CA (US)

(21) Appl. No.: 14/137,766

(22) Filed: Dec. 20, 2013

Related U.S. Application Data
(60) Provisional application No. 61/740,171, filed on Dec. 20, 2012.

(51) Int. Cl. A61B 17/3205

(52) U.S. Cl. CPC A61B 17/3205
USPC 606/180

(57) ABSTRACT

A tissue removal device may comprise a handle portion and a tissue removal mechanism coupled to the handle portion. The tissue removal mechanism may include a tubular member having a lumen therethrough and an elongate member rotatably and slidably disposed within the lumen of the tubular member. A proximal end of the elongate member may be coupled to a drive source to impart rotational movement thereof. A distal end of the elongate member may include an impeller for cutting tissue. The elongate member may be configured to exit the distal end of the lumen of the first tubular member, such that the distal end of the second tubular member is distal to the distal end of the first tubular member. The impeller may include a blunt distal end to minimize undesirable tissue damage when the distal end of the elongate member is distal to the distal end of the tubular member.

This application claims priority to U.S. Provisional Application Ser. No. 61/740,171, entitled “Discectomy Devices and Methods,” filed Dec. 20, 2012, which application is incorporated herein by reference in its entirety.

### BACKGROUND

Vertebral disc herniation is a common disorder where a portion of a vertebral disc, a cushion-like structure located between the vertebral bodies of the spine, bulges out or extrudes beyond the usual margins of the disc and the spine. Disc herniation is believed to be the result of excessive loading on the disc in combination with weakening of the annulus due to such factors as aging and genetics. Disc herniation and other degenerative disc diseases are also associated with spinal stenosis, a narrowing of the bony and ligamentous structures of the spine. Although disc herniation can occur anywhere along the perimeter of the disc, it occurs more frequently in the posterior and posterior-lateral regions of the disc, where the spinal cord and spinal nerve roots reside. Compression of these neural structures can lead to pain, parasthesias, weakness, urine and fecal incontinence and other neurological symptoms that can substantially impact basic daily activities and quality of life.

Temporary relief of the pain associated with disc herniation is often sought through conservative therapy, which includes positional therapy (e.g. sitting or bending forward to reduce pressure on the spine), physical therapy, and drug therapy to reduce pain and inflammation. When conservative therapy fails to resolve a patient's symptoms, surgery may be considered to treat the structural source of the symptoms. Surgical treatments for disc herniation traditionally involve open procedures that involve dissection of muscle, connective tissue and bone along a patient's back as well as nerve manipulations to achieve adequate surgical exposure. For example, a discectomy procedure may be used to decompress the herniation by accessing the affected disc and removing a portion of the disc and any loose disc fragments. In some cases, a portion of the lamina or bony arch of the vertebrae may be removed. When discectomy fails to resolve a patient's symptoms, more drastic measures may include disc replacement surgery or vertebral fusion.

### BRIEF SUMMARY

Consistent with the present disclosure, a device may comprise a handle and a tissue removal mechanism coupled to the handle. The tissue removal mechanism may include a tubular member having a lumen therethrough terminating in a distal opening and an elongate member rotatably and slidably disposed within the lumen of the tubular member. A proximal end of the elongate member may be coupled to a drive source to impart rotational movement thereof. A distal end of the elongate member may include an impeller, the elongate member being configured to advance from the distal opening of the tubular member, such that a portion of the impeller is distal to the distal end of the tubular member.

In certain variations, the impeller may include a blunt distal tip. In other variations, the tissue removal mechanism may further comprise a tissue collection chamber coupled to the lumen of the tubular member. In still other variations, the tissue removal mechanism may further include a helical member disposed about a portion of the elongate member.

In certain variations, the tubular member may include a distal tip having an opening. The opening may be formed by, or otherwise include, a protrusion, while in other variations the protrusion is a first of a plurality of protrusions. The plurality of protrusions may form a serrated edge. In other variations, the opening includes an edge, and the impeller may be configured to cooperate with the edge to facilitate tissue removal. Such edge may be sharpened, for example.

In certain variations, the impeller may include a helical flute. The helical flute may be configured to cooperate with the lumen to break up target tissue into a plurality of target tissues, each of smaller size for example. In other variations, the helical flute may be configured to cooperate with the distal opening of the tubular member to further facilitate tissue removal, when a distal tip of the impeller is advanced out the distal opening of the tubular member for example.

In certain variations, the elongate member includes a distal end including an angled surface, which may assist in dilating surrounding tissues. In still other variations, the drive source may be an electric motor. A power source configured to supply power to the drive source may be located in the handle. In certain other variations, the elongate member may comprise the impeller and a tubular member having a distal end coupled to the impeller and a proximal end coupled to the drive source.

In certain variations, a method may include advancing the device to a target tissue, and advancing the elongate member such that a portion of the impeller is distal to the distal opening of the tubular member. The method may also include removing a portion of the target tissue with the tissue removal mechanism while the portion of the impeller is distal to the distal opening of the tubular member. In other variations, removing the portion of the target tissue with the tissue removal mechanism may include removing a portion of target tissue with the portion of the impeller that is distal to the distal opening of the tubular member. In still other variations, the tissue removal mechanism may further comprise a tissue collection chamber, and the method may include gathering the portion of the target tissue in the tissue collection chamber. In other certain variations, the portion of the target tissue may be a first portion and the opening of the tubular member may be a first opening, and the tubular member may include a second opening, and the method may include removing a second portion of the target tissue through the second opening of the tissue removal mechanism.

### BRIEF DESCRIPTION OF THE DRAWINGS

FIG. 1 is a schematic perspective view of a portion of a lumbar spine.

FIG. 2 is a schematic superior view of a portion of a lumbar vertebra and disc.

FIG. 3A is a schematic lateral view of a portion of a lumbar spine (without the spinal nerves), and FIG. 3B depicts the portion of the lumbar spine in FIG. 3A (with the spinal nerves depicted).

FIG. 4 is a side view of an exemplary discectomy device, in accordance with certain aspects of this disclosure.

FIG. 5A is a detailed side view of a distal portion of the exemplary discectomy device of FIG. 4; and FIG. 5B is an exemplary cutter which may be employed with the exemplary discectomy device of FIG. 4.

FIG. 6 is a side view of the discectomy device of FIG. 4, where the exemplary cutter is in an extended configuration.

FIG. 7A is a sectional view of the distal portion of the exemplary discectomy device of FIG. 4; and FIG. 7B is a detailed view of an exemplary auger which may be employed with the exemplary discectomy device of FIG. 4.

FIG. 8 is a sectional view of the discectomy device of FIG. 4, where the exemplary cutter is in an extended configuration.

### DETAILED DESCRIPTION

Tissue removal devices and methods, such as discectomy devices and methods, are described herein. In certain variations, a discectomy device may be introduced into a disc via dilation of an access hole through the annulus, such that it may not be necessary to cut the annulus to access the disc. In some variations, a discectomy device may comprise a relatively long auger, and/or an impeller that breaks down acquired tissue during a procedure. During use, the auger and impeller may effect a plunging motion that allows for relatively rapid tissue aspiration and aggressive tissue cutting, without stretching the annulus. Additionally, it may not be necessary to make several passes into and out of a patient to remove tissue, using devices and methods described herein. By limiting cutting, stretching and/or the number of passes through tissue, scarring of annular tissue, reherniation and/or leakage of healthy nucleus tissue may be avoided, and/or annulus healing time may be reduced.

In some cases, devices described herein may be capable of breaking down soft tissue and/or relatively tough, hardened nucleus tissue, and/or may be used to aspirate different types of tissue varying in consistency, hardness and/or elasticity. In some variations, devices described herein may be used to cut hard tissue, such as bone. In some cases, the cant angle of a device's cutting edge or edges (e.g. between the inner base surface of the impeller and the cutting edge of the impeller) may be adjusted to differentially cut relatively hard or calcified materials or tissues without also cutting relatively soft materials or tissues.

In some variations, to be the least destructive to spine structures while preserving the strength of the bones, a spinal procedure may be minimally invasive while also reducing the amount of excised, native bone or dissection of surrounding native tissues. Minimally invasive tissue removal devices may, for example, be configured for insertion toward or into a vertebral disc without requiring suturing, gluing or other procedures to seal or close the access pathway into the disc. The exemplary variations described herein include but are not limited to minimally invasive devices or systems and methods for performing discectomies and other tissue removal procedures, as appropriate. For example, a microdiscectomy may be performed using one or more of the devices and/or methods described herein.

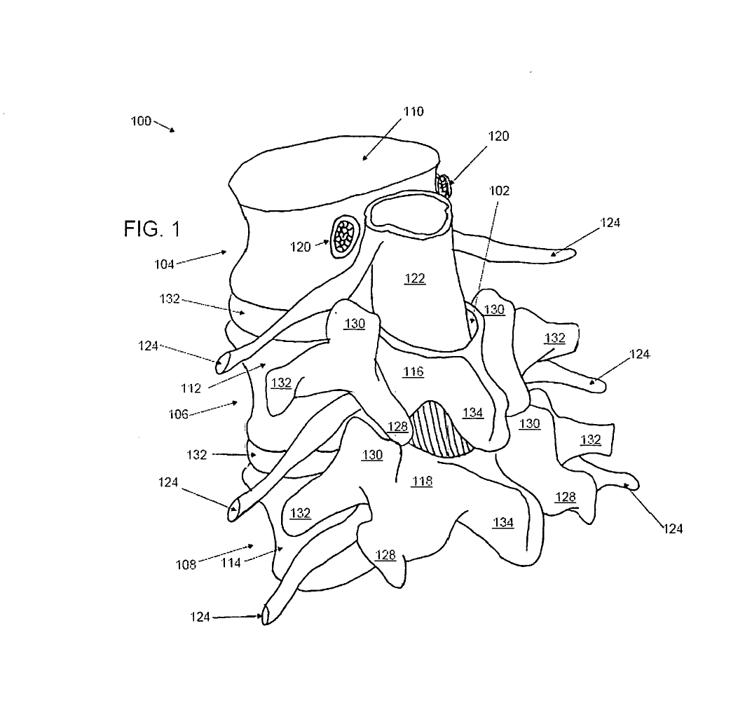

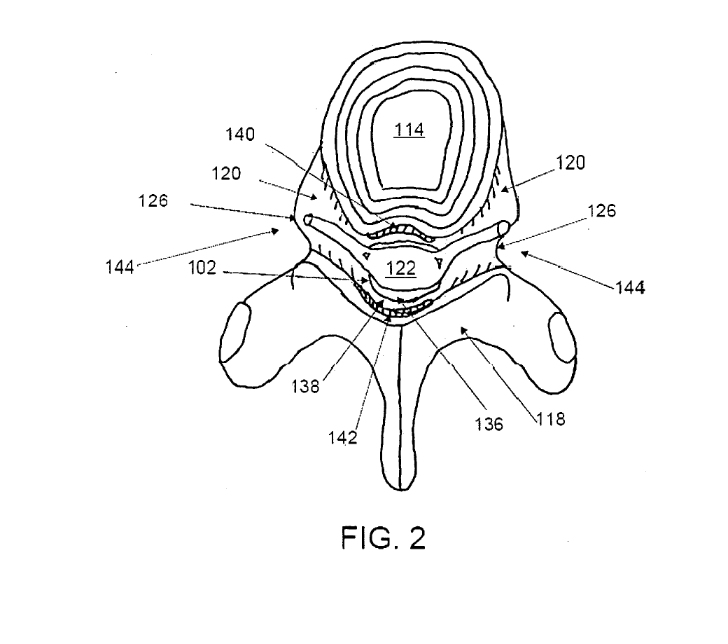

FIG. 1 is a schematic perspective view of a lumbar portion of a spine 100. The vertebral canal 102 is formed by a plurality of vertebrae 104, 106, and 108, which comprise vertebral bodies 110, 112, and 114 anteriorly and, with respect to vertebral bodies 112, 114, vertebral arches 116 and 118 posteriorly. The vertebral arch and adjacent connective tissue of the superior vertebra 104 in FIG. 1 has been omitted to better illustrate the spinal cord 122 within the vertebral canal 102. Spinal nerves 124 branch from the spinal cord 122 bilaterally and exit the vertebral canal 102 through intervertebral foramina 126 that are formed between adjacent vertebra 104, 106 and 108. The intervertebral foramina 126, as better viewed with respect to FIG. 2, are typically bordered by the inferior surface of the pedicles 120, a portion of the vertebral bodies 104, 106 and 108, the inferior articular processes 128, and the superior articular processes 130 of the adjacent vertebrae. Also projecting from the vertebral arches 116 and 118 are the transverse processes 132 and the posterior spinous processes 134 of the vertebrae 106 and 108. Located between the vertebral bodies 110, 112 and 114 are vertebral discs 134.

Referring to FIG. 2, the spinal cord 122 is covered by a thecal sac 136. The space between the thecal sac 136 and the borders of the vertebral canal 102 is known as the epidural space 138. The epidural space 138 is bound anteriorly and posteriorly by the longitudinal ligament 140 and the ligamentum flavum 142, respectively, of the vertebral canal 102, and laterally by the pedicles 120 of the vertebral arches 116 and 118 and the intervertebral foramina 126. The epidural space 138 is contiguous with the paravertebral space 144 via the intervertebral foramina 126.

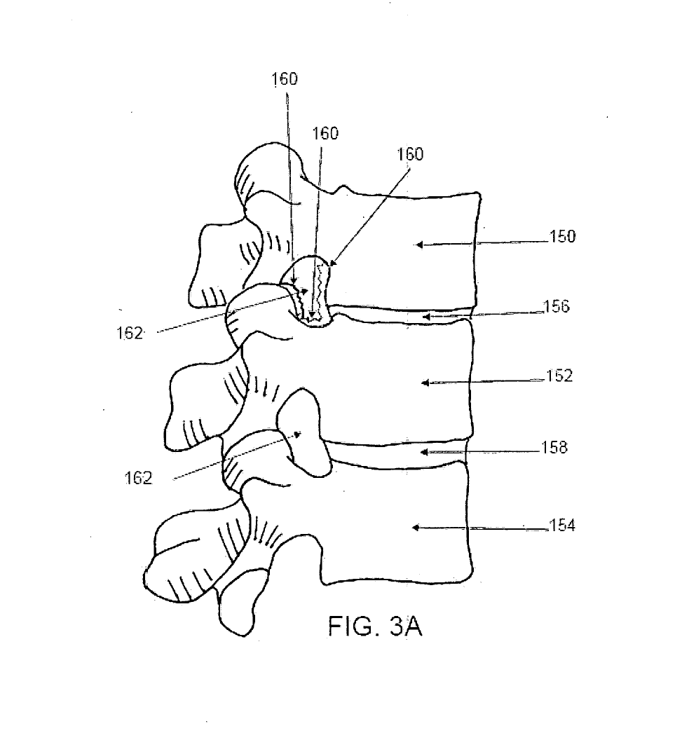

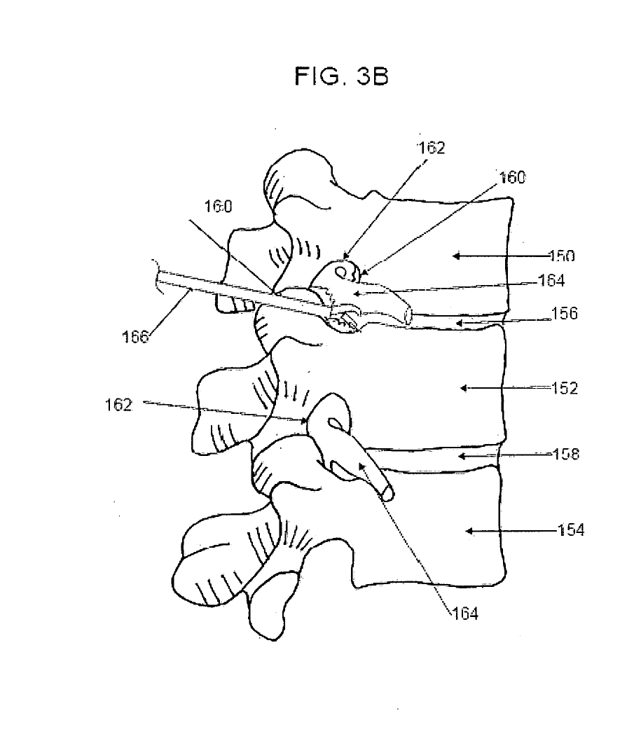

With degenerative changes of the spine, which include but are not limited to disc bulging and hypertrophy of the spinal ligaments and vertebrae, the vertebral canal 102 may narrow and cause impingement of the spinal cord or the cauda equina, a bundle nerves originating at the distal portion of the spinal cord. Disc bulging or bone spurs may also affect the spinal nerves 124 as they exit the intervertebral foramina 126. FIG. 3A, for example, schematically depicts a lateral view of three vertebrae 150, 152 and 154 with intervertebral discs 156 and 158, without the spinal cord or spinal nerves. With degenerative changes, regions of bone hypertrophy 160 may develop about the intervertebral foramina 162. While secondary inflammation of the associated nerve and/or soft tissue may benefit from conservative therapy, the underlying bone hypertrophy remains untreated. The regions of bone hypertrophy 160 may be removed, with or without other tissue, using open surgical spine procedures, limited access spine procedure, percutaneous or minimally invasive spine procedures, or combinations thereof. FIG. 3B depicts the vertebrae 150, 152 and 154 of FIG. 3A with their corresponding spinal nerves 164 during a foraminotomy procedure using a burr or grinder system 166.

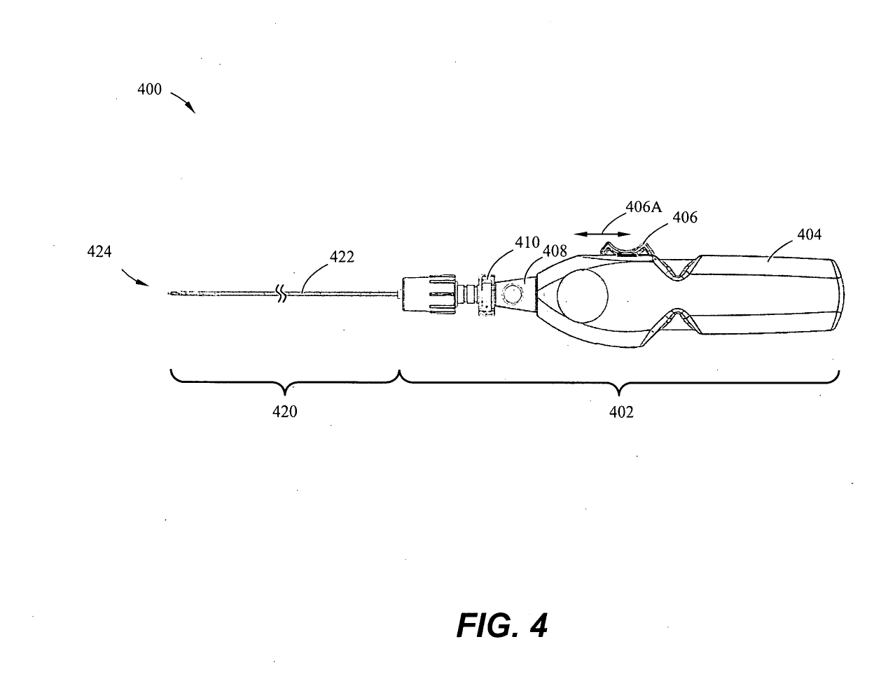

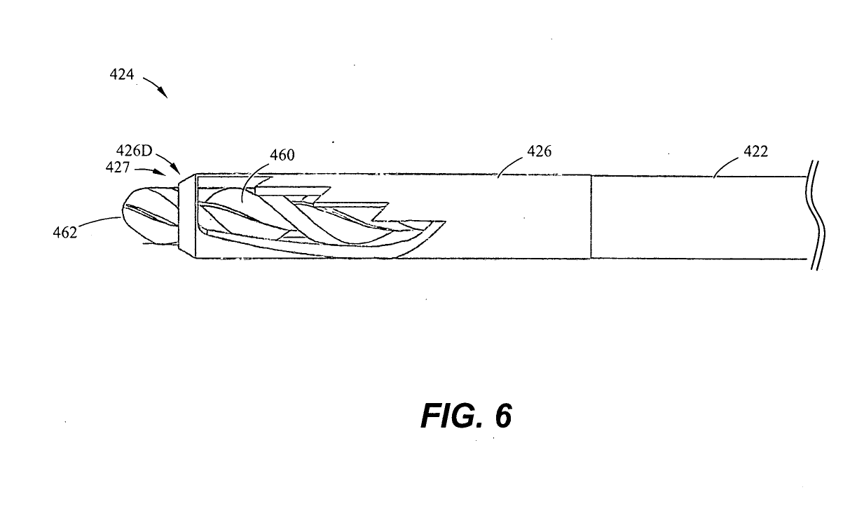

FIG. 4 depicts one variation of a tissue removal device 400, comprising a handle portion 402 and a tissue removal mechanism 420. The handle portion 402 includes a handle 404, a slider 406 control, a tissue collection chamber 408, and a travel limiter 410. The tissue removal mechanism includes a tubular member 422 and a tissue removal assembly 424, the tubular member 422 extending from a distal end of the handle portion 402 to the tissue removal assembly 424. The handle 404 may be fabricated from any suitable material and may include one or more surface features, such as indentions, or recessed or grooved regions, to allow for easier control and manipulation by a doctor or surgeon, otherwise referred to herein as a practitioner or user, during use. The slider 406 may be actuated in the directions indicated by arrow 406A for operation of one or more features of the tissue removal device 400, as described in greater detail below with respect to FIG. 6. As is described in greater detail below, the slider 406 may regulate the configuration and use of the tissue removal assembly 424. Other handle variations may comprise one or more push buttons, sliders, dials, or knobs, for controlling elements of the tissue removal assembly 424 for example.

The tissue collection chamber 408 is coupled to the tissue removal assembly 424 of the tissue removal mechanism 420 such that tissue may be collected in collection chamber 408 during operation of the tissue removal device 400. While shown being attached to a distal end of the handle 404, the collection chamber 408 may be located elsewhere with respect to the handle, or may be separately attached to a port or conduit of the handle. The travel limiter 410 may cooperate with other structures of the tissue removal device 400 (not shown) through which the tissue removal mechanism 420, or a portion thereof, translates, the operation of a similar device is described, for example, in copending patent application Ser. No. 12/753,788, entitled “Minimally Invasive Discectomy” (“'788 Application”), which is hereby incorporated by reference in its entirety.

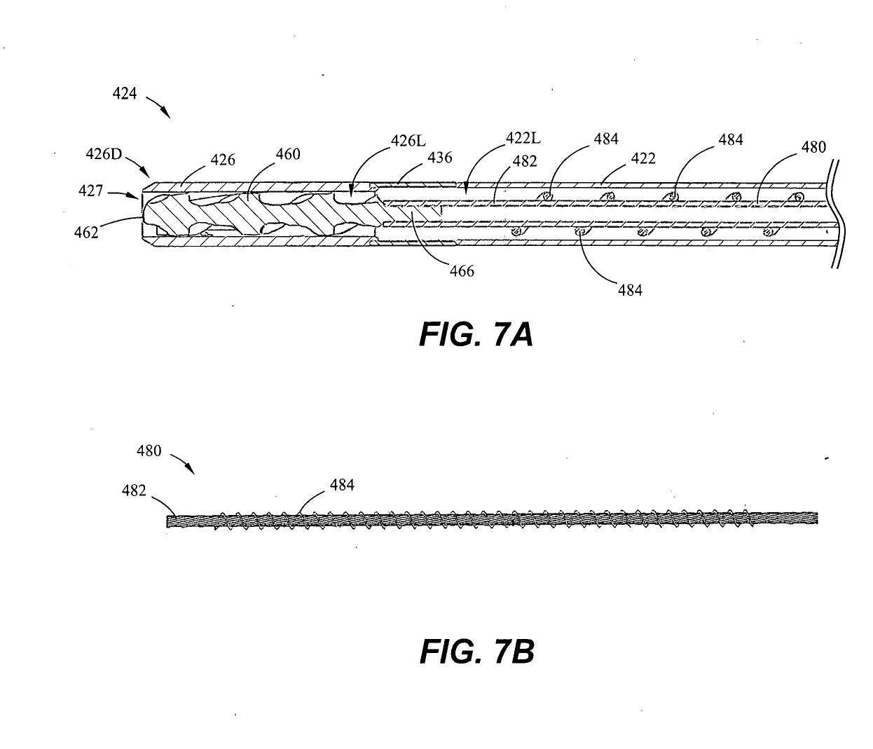

The tubular member 422 may be used to provide a conduit between the tissue removal assembly 424 and the collection chamber 408 and/or handle 404 via a longitudinal lumen therethrough, as is described in greater detail below with respect to FIG. 7A. The tubular member 422 may be flexible, steerable, deformable, and/or bendable, as appropriate for directing the distal tissue removal assembly to the target tissue. Different flexibilities and curvatures of the outer tube may help the tissue removal device to access spinal and/or vertebral tissue, or another particular region of the body. For example, the tubular member 422 may have one or more malleable or flexible regions along its length, which may provide additional maneuvering capability to a practitioner. While the tubular member 422 is depicted to be substantially straight, in other variations, the tubular member 422 may have one or more pre-shaped curves, where the curves may be substantially rigid or substantially flexible. For example, a straight or curved access pathway to the target tissue may be additionally adjusted and/or shaped by the curvature of the tubular member 422. In some variations, access to a target tissue may be provided through a straight or curved cannula through which the tubular member 422 translates. A tubular member 422 with one or more flexible curved regions may be straightened by sliding it into a straight cannula, or flexed by sliding it into a curved cannula. Alternatively, tubular member 422 with rigid curved regions may be inserted into a bendable flexible cannula and cause it to curve along the curved regions. In other variations, the tubular member 422 may be flexed or otherwise manipulated using a steering mechanism, an example of which is described in the '788 Application.

The tubular member 422 may be a hypotube or a multifilament braided or coiled cable. The tubular member 422 may be made of a metal such as 304 stainless steel, a metal alloy such as nickel titanium alloy, or a polymer, such as polyimide, or a combination thereof, and may comprise any of a variety of structural configurations. For example, the tubular member 422 may comprise a braided or extruded polyimide. In certain variations of the tubular member 422, all or portions of the tubular member 422 may be coated with an additional material to help prevent galling effects, and/or to provide thermal insulation, which may help prevent thermal damage to tissue structures. Additionally, the tubular member may further include various visual enhancements to improve visibility and placement thereof, with an endoscopic camera system or fluoroscopy system for example, during use.

The handle portion 402 contains one or more components configured to control the tissue removal assembly 424 and other optional features of the tissue removal device 400. The tubular member 422 includes a lumen that encases a rotating drive shaft (as shown in FIG. 7A) that is coupled to the tissue removal assembly 424. Tissue removal assemblies such as tissue removal assembly 424, may be configured to grasp, cut, chop, grind, burr, pulverize, debride, debulk, emulsify, disrupt or otherwise remove tissue, as appropriate. Emulsification includes, for example, forming a suspension of tissue particles in a medium, which may be the existing liquid at the target site, liquid added through the tissue removal device, and/or liquid generated by the debulking of the tissue. Optional components of tissue removal device 400 and other tissue removal devices described herein may include, but are not limited to, a motor configured to rotate or move one or more components of the tissue removal assembly 424, a power source or power interface, a motor controller, a tissue transport assembly (e.g. comprising an auger), an energy delivery or cryotherapy assembly, a therapeutic agent delivery assembly, a light source, and one or more fluid seals. The optional tissue transport assembly may comprise a suction assembly and/or a mechanical aspiration assembly. One or more of these components may act through the tubular member 422 to manipulate the tissue removal assembly 424 and/or other components located distal to the handle portion 402, or from the handle 404 directly. For example, the tissue removal device 400 may further comprise an optional port that may be coupled to an aspiration or suction source to facilitate transport of tissue or fluid out of the target site or patient. The suction source may be a powered vacuum pump, a wall suction outlet, or a syringe, for example.

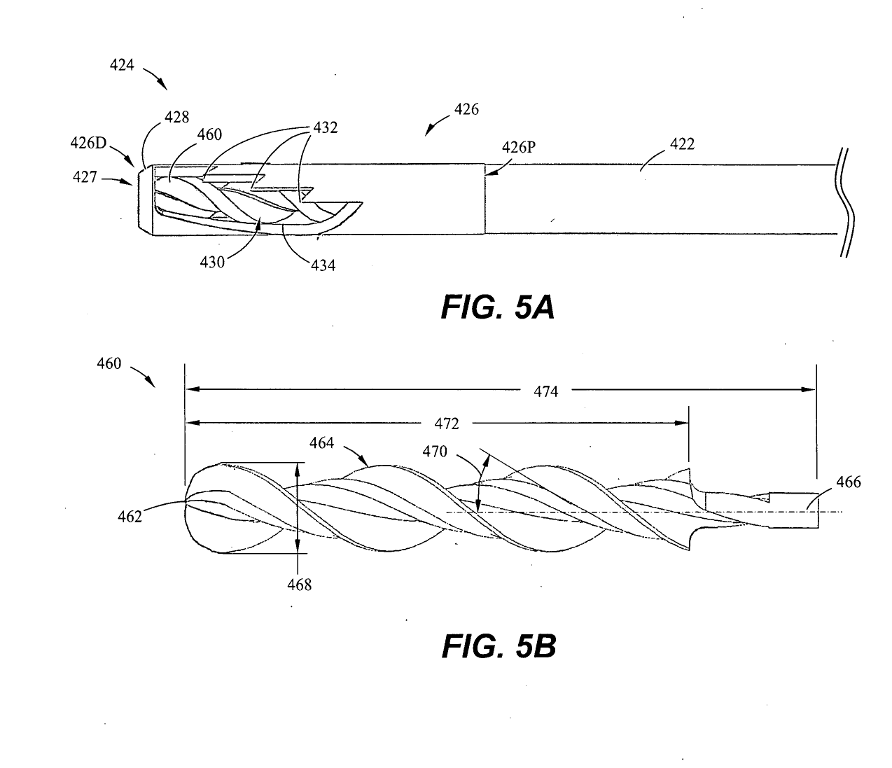

Turning now to FIG. 5A, the tissue removal assembly 424 will be described in greater detail. The tissue removal assembly 424 may include a tip 426 and an impeller 460. The tip 426 includes a proximal portion 426P and a distal portion 426D, and a lumen therethrough, the lumen being coupled to a lumen of the tubular member 422, as described in greater detail below with respect to FIG. 7A. The lumen of the tip 426 extends out an opening 427 at the distal end 426D, allowing the impeller 460 to extend through the opening and engage target tissue more distal to the tip 426. The distal end 426D may include angled surfaces 428, as shown, to assist in the dilation of surrounding tissue during use. The tip 426 may include one or more lateral windows or openings 430 extending along a length of the tip 426, as generally shown. The configuration of such openings 430 may be such as to encourage tissue removal. For example, the opening may be formed, in part, by one or more protrusions 432, which together may form a serrated edge. Further, the edges forming the opening 430, such as edge 434, may cooperate with the impeller 460 to better facilitate tissue removal, the target tissue being cut there between.

Referring now to FIG. 5B, the impeller 460 may include a helical flute 464 configured to cooperate with the lumen of the tip 426 to break target tissue into smaller portions and/or transport such target tissue through the lumen of the tip 426 and, ultimately, into the tissue collection chamber 408, for example. The impeller 460 includes a blunt tip 462 to limit the forward cutting abilities such that inadvertent movement into tissue adjacent to the target tissue does not result in undesirable tissue damage, preventing inadvertent cutting into a vertebral end-plate or an annulus during use for example. The impeller 460 includes a proximal portion configured to interface with a rotating tubular member, described in greater detail below with respect to FIG. 7A.

Impeller 460 may have dimensions 468, 470, 472 and 474, and such dimensions may be selected as needed and only limited by manufacturability. For illustration purposes only, dimension 474 may be from about 0.3 inch to about 0.8 inch, dimension 472 may be from about 0.2 inch to about 0.5 inch, dimension 468 may be from about 0.02 inch to about 0.08 inch, and/or dimension 470 may be from about 25 degrees to about 45 degrees. Impeller 460 may, for example, have a pitch (or distance between two adjacent revolutions of the impeller) of about 0.2 inch to about 0.4 inch. Impellers may be made of any appropriate material or materials, including but not limited to metals and/or metal alloys such as stainless steel (e.g. 17-4 PH H900 stainless steel). Other variations of impellers having different configurations may also be used. Such impellers, for example, may be disclosed in the '788 Application, or in pending U.S. patent application Ser. No. 13/396,438, entitled “Discectomy Devices and Related Methods” (“'428 Application”), and U.S. patent application Ser. No. 13/560,850, entitled “Discectomy Devices and Related Methods”, both of which are incorporated herein in their entirety by reference (“'850 Application”). It should be understood that features of the above-described impeller 460 and/or other components described herein may be applied to other impellers and/or components of tissue removal devices, as described in the '788 Application, the '428 Application or the '850 Application, for example.

Turning to FIG. 6, the tissue removal assembly 424 is depicted with the impeller 460 in an extended configuration, for example the blunt distal end 462 of the impeller 460 extending through opening 427 in the distal end 426D of the tip 426. The slider 406 of handle assembly 402, as depicted in FIG. 4, may be coupled to the tissue removal assembly 424 and employed to extend the impeller 460 past the distal end 462D of tip 426. For example, sliding the slider 406 in a distal direction may result in the movement of the blunt tip 462 of impeller 460 to a position distal to the distal end 426D of tip 426 of the tissue removal assembly 424, while sliding the slider 406 in a proximal direction may result in the movement of the tip 462 of impeller 460 to a position proximal to the distal end 426D of tip 426. The impeller may extend out the opening 427 of the distal end 426D of the tip 426 any suitable distance. For illustration purposes only, the tip 462 of impeller 460 may exit and travel up to about 5 mm past the opening 427 of the distal end 426D of the tip 426.

Now turning to FIG. 7A, a portion of the tissue removal mechanism 420, including the tissue removal assembly 424, is shown in cross-section. As shown, the tubular member 422 includes a lumen 422L, which is in fluid communication with a lumen 426L of the tip 426. A rotatable member 480, which may or may not be tubular, includes a proximal end (not shown) coupled to a drive source, such as an electric motor as described in the '788 Application, for example, and a distal end 482 coupled to the proximal end 466 of the impeller 460. The rotatable member 480 may be coupled to the impeller 460 through any suitable means, such as via a mechanical compression fit, soldering or brazing, or through the use of a suitable and compatible adhesive for example, such that rotation of the tubular member 480 imparts rotation of the impeller 460. The tip 426 may be coupled to tubular member 422 through any suitable means, such as via a mechanical compression fit, soldering or brazing, or through the use of a suitable and compatible adhesive for example, or through the use of a coupling member 436, which aides in the coupling of the tubular member 422 to the tip 426. For illustration purposes only, the coupling member 436 may be a ring that is compressed upon a joint between the tubular member 422 and the tip 426, or may be adhered in place through the use of a suitable and compatible adhesive. Alternatively, while shown and described herein as separate items, the tip 426 may be an integral part of the tubular member 422.

With reference also to FIG. 7B, the rotatable member 480 may include a helical portion 484 about the outer diameter of the rotatable tubular member 482 to assist in the transportation of collected target tissue from the impeller 460 to the tissue collection chamber 408. The helical portion 484 may be part of the rotatable tubular member 482 or may be fabricated, for illustration purposes, from a wire wrapped about the rotatable member 480 and fixed thereto through soldering, brazing, or application of a suitable and compatible adhesive. With the helical portion 484, the rotatable member 480 may act as, and may be, an auger 480. Although auger 480 is depicted as a continuous structure, in some variations, auger 480 may be interrupted at one or more locations along its length. Also, for illustration purposes only, the degree or angle of tightness of the helical pattern of the auger 480 may vary, from about 0.5 turns/mm to about 2 turns/mm, sometimes about 0.75 turns/mm to about 1.5 turns/mm, and other times about 1 turn/mm to about 1.3 turns/mm. The cross-sectional shape of the auger 480 may be generally rounded, but in other variations, may have one or more edges. The general cross-sectional shape of the auger 480 may be circular, elliptical, triangular, trapezoidal, squared, rectangular or any other shape. The turn tightness and cross-sectional shape or area of the auger 480 may be uniform or may vary along its length.

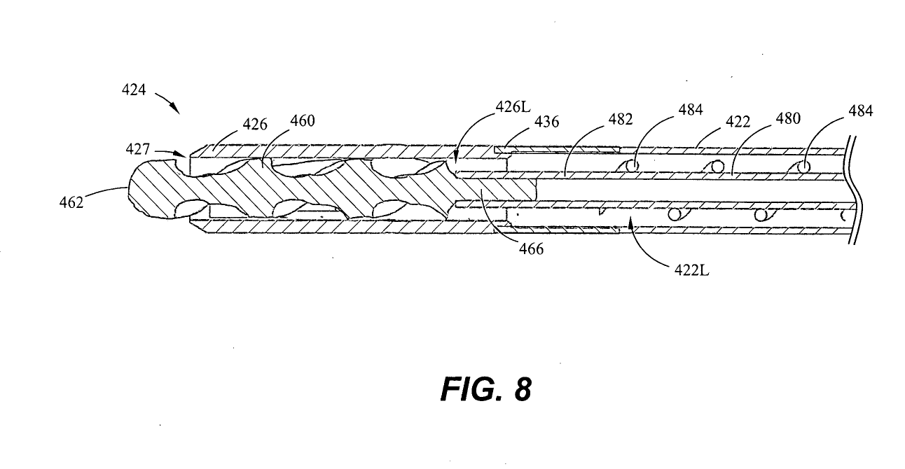

Turning to FIG. 8, a portion of the tissue removal mechanism 420 is depicted in cross-section with the impeller 460 in an extended configuration, for example the blunt distal end 462 of the impeller 460 extending through an opening 427 at the distal end 426D of the tip 426. The slider 406 of handle assembly 402, as depicted in FIG. 4, may be coupled to the tissue removal assembly 424 and employed to extend the impeller 460 past the distal end 462D of tip 426. For example, sliding the slider 406 in a distal direction may result in the movement of the blunt tip 462 of impeller 460 to a position distal to the distal end 426D of tip 426 of the tissue removal assembly 424, while sliding the slider 406 in a proximal direction may result in the movement of the tip 462 of impeller 460 to a position proximal to the distal end 426D of tip 426, as depicted in FIG. 5A for example.

The various tissue removal devices disclosed herein may be used to perform a discectomy or nucleotomy, but may also be used to perform any of a variety of tissue removal procedures in the spine and outside of the spine. Examples of procedures that may be used to access the spine are disclosed in U.S. Pat. No. 7,108,705, U.S. Pat. No. 4,573,448, U.S. Pat. No. 6,217,5009, and U.S. Pat. No. 7,273,468, which are hereby incorporated by reference in their entirety.

The tissue removal devices may be used in minimally invasive procedures as well as open surgical procedures or limited access procedures. These procedures may include but are not limited to interlaminar, translaminar and intralaminar access procedures. In one particular embodiment, a patient may be placed into a prone position with a pillow or other structure below the abdomen to limit lumbar lordosis. The patient may be prepped and draped in the usual sterile fashion and anesthesia may be achieved using general, regional or local anesthesia. Under fluoroscopic guidance, a sharp tipped guidewire, or a needle with a guidewire, may be inserted into the paravertebral space or epidural space from a posterior or postero-lateral location of the patient's back at a location in the range of about 2 inches to about 6 inches lateral to the midline. In some instances, guidewire insertion may be facilitated by inserting a needle into the tissue first. In alternate variations, an anterior procedure through the abdominal cavity or anterior neck region may be performed. Once access to the target location is confirmed, a dilator may be used with the guidewire to enlarge the insertion pathway. Then, an introducer or cannula may be inserted over the guidewire, followed by subsequent guidewire removal and insertion of an endoscope into the introducer or cannula. Alternatively, an endoscope may be inserted over the guidewire. The endoscope may be manipulated or steered to directly visualize and identify the relevant structures such as the disc, the nerve or other adjacent structures and site(s) of tissue removal. In some variations where the patient is under local or regional anesthesia, a suspected nerve impingement may be confirmed by contacting or manipulating the suspected nerve with the endoscope, or other device inserted through the endoscope, and assessing the patient's response or symptoms. One variation of an endoscope that may be used is described in U.S. patent application Ser. No. 12/199,706, which is hereby incorporated by reference in its entirety.

Once the target region has been evaluated, a tissue removal device may be inserted through the spinal access device or endoscope and to pierce through the annular wall of a herniated disc. Once inserted, the tissue removal device may be manipulated and actuated to remove the target tissue. In some variations, the tissue removal device may be actuated, for example resulting in the rotation of impeller 460, with or without translation of the blunt tip 462 past the distal end 426D of tip 426, for a duration in the range of about 5 seconds to about 90 seconds or more, sometimes about 15 seconds to about 60 seconds, and other times about 30 seconds to about 60 seconds.

In certain variations, any collected material may be suctioned through the device and then the effect of the tissue removal may be re-evaluated by the endoscope or other visualization mechanisms. In some variations, a liquid or lubricant may be injected or infused into the treatment site. In some examples, the liquid or lubricant may be useful to facilitate removal of the collected material, including but not limited to vertebral discs that may be desiccated. In other examples, the liquid or lubricant may be injected or infused before or during the actuation of the tissue removal device. In some examples, the liquid or lubricant may comprise a contrast agent that may facilitate viewing of the tissue site on fluoroscopy, x-ray, CT, MRI, ultrasound or other imaging modalities. The contrast agent may be used at any time or at multiple times during the procedure, including but not limited to confirmation of guidewire or tissue removal device placement, and also to verify the volume and/or location of tissue removal.

In some specific variations, actuation of the tissue removal device may be stopped to verify that the annulus of the vertebral disc or the cortical bone of the vertebral body has not been compromised. Also, in some examples, contrast agent may be injected and imaged after device actuation to assess proper operation of the device, including but not limited to tissue pulverization and aspiration mechanisms.

During actuation, the tissue removal device may be held in place or may be moved around the treatment site. Suction or aspiration may be applied during these motions to assess the amount of tissue being removed.

The actuation of the tissue removal device may be repeated as desired to remove disc material. In some embodiments, the tissue removal device may be withdrawn from the disc and reinserted directly into or against the extruded disc material and actuated. Once the tissue removal is completed, the tissue removal device may be withdrawn. The puncture site in the annular wall may have a cross-sectional area of less than about 0.003 inch² or less, sometimes about 0.0016 inch² or less, and other times about 0.001 inch² or less, and thus may self-seal without requiring treatment of the puncture location with an adhesive, a suture or coagulation probe. The body location may be rechecked with the endoscope or spinal access device to verify that no bleeding or compromise of the integrity of the disc or spinal nerves has occurred, and then the endoscope or spinal access device may be removed from the body and the skin access site may be bandaged.

While various tissue removal devices may be used to remove larger volumes of tissue, in other variations, a tissue removal device may be used to perform focal debulking of tissue. For example, by utilizing the small profile and/or the steerable features of certain variations of the tissue removal device, the tissue removal device may be more accurately positioned or navigated to a specific target site in a body structure. In some instances, the removal of lower volumes of tissue at a specific target location may be used to achieve a desired result, in comparison to the removal of a larger volume of tissue from a general target location. By removing less disc tissue to reduce a herniation, for example, a larger amount of non-pathologic disc tissue and structural integrity of the disc may be preserved. In some instances, relatively greater preservation of the disc tissue may slow the rate of further disc degeneration and reherniation compared to lesser degrees of tissue preservation.

In one example, a herniated disc may be accessed and visualized endoscopically. A steerable tissue removal device may be inserted into the disc and steered toward the region of herniation, rather than to the center of the disc, for example.

The procedures described herein may target vertebral tissue in different locations, and as such, access sites and pathways may vary accordingly. The tissue removal devices described above may be used with one or more access devices that may help direct the tissue removal device to the target tissue site. An access device, such as a cannula, may be positioned with different angles of entry depending on the location of the targeted vertebral tissue. The range of suitable entry angles may be at least partially constrained by the location of spinal structures with respect to the skin surface. For example, a straight cannula may be positioned within the range of suitable entry angles to create a linear access pathway that extends from an access site on the skin surface to a targeted region of spinal tissue that is co-linear with access site. A curved cannula may be used to create a curved pathway to access tissue that may not be co-linear with an access site within a suitable entry angle range. While a curved pathway may provide increased accessibility to vertebral tissue, a practitioner may need to undergo additional training and practice to avoid disrupting sensitive anatomical structures along a curved pathway. Some variations of access devices may comprise a bendable flexible curvable cannula, which may have a straight configuration and a curved configuration. The cannula may be used in the straight configuration to create a substantially linear access pathway from the access site on the skin surface to the vicinity of the target vertebral tissue. Once the initial access pathway is created, the cannula may be used in the curved configuration to contact the target tissue.

In some variations, the curvature of a cannula may be determined in part by the curvature of a stylet inserted therethrough. For example, inserting a stylet with one or more curves into a bendable flexible cannula may cause the cannula to have corresponding curves. In some variations, a bendable cannula may have one or more pre-formed curves that may be straightened by inserting a straight stylet therethrough. Alternatively, a bendable cannula that is substantially straight may be curved by inserting a curved stylet therethrough. The insertion of various stylets through a bendable cannula may allow a practitioner to access spinal tissue at different locations via one access site on the skin. This may reduce the need for withdrawing the cannula from the body and re-entering the body via an additional access site to access a different tissue region. For example, the cannula and the stylet may each have one or more corresponding curves such that when the stylet is inserted through the cannula, the corresponding curves may be aligned. This may act to stiffen or reinforce the curvature of the cannula so that it may be more easily moved from a first tissue location to a second tissue location. For example, a procedure performed on one tissue location in the disc annulus may be repeated at another tissue location without removing the curved cannula from the disc annulus. While at the first tissue location, a curved or straight stylet may be reintroduced into the cannula, which may facilitate adjustment and positioning of the cannula to a second tissue location. Insertion of a straight stylet may straighten the curved portion of the cannula and allow the cannula-stylet assembly to be advanced to a target site that is relatively further away from the site that has been treated. In other embodiments where relatively insignificant cannula repositioning is involved, a curved stylet may be used to acquire access to a second target site within the disc. A straightened and/or stiffened cannula-stylet assembly may offer enhanced responsiveness and maneuverability and therefore facilitate the maneuvering of the cannula within the discal area, and may facilitate safe removal of the devices from a patient.

The length of a stylet may be greater than, or substantially equal to the length of a corresponding cannula. For example, the distal portion of a stylet inserted into a cannula may extend or protrude from the distal portion of the cannula, and/or may be flush with the distal portion of the cannula, and/or may even be withdrawn into the cannula, as desirable. Similarly, the tissue removal assembly of a tissue removal device may be extended from and/or withdrawn into the distal portion of the cannula. The relative longitudinal position between a cannula and stylet, and/or cannula and a travel limiter of a tissue removal device may be adjusted and/or locked. In some variations, the orientation of one or more curves in a cannula and a stylet with respect to each other may be adjusted by rotating the stylet, and may optionally be locked once the desired orientation is obtained. The cannula and stylet may each comprise complementary proximal connectors, which may be used to couple them together, such that they may be advanced and navigated together. Optionally, the proximal connectors may rotatably and/or longitudinally lock the cannula and stylet with respect to each other.

Some variations of a cannula and/or stylet may have an orientation indicator, which may help a practitioner to identify the orientation of the one or more curves of the devices, or the orientation of one or more sharpened edges of a stylet, after they have been inserted into the body of a patient. For example, the orientation of a distal curve of a cannula with respect to the longitudinal axis of the cannula shaft may be evident by observing the configuration of the orientation indicator. Orientation indicators may also help a practitioner align the curvature of a stylet to correspond with the curvature of the cannula that it is inserted through. In this way, the practitioner may proximally adjust the bend orientation of the stylet, thereby allowing the stylet to pass through the cannula bend with ease. The shape of the orientation indicator may convey the orientation of the one or more curves of the cannula and/or style to the practitioner. For example, the orientation indicator may have a shape with one or more tapered regions, where the plane of a taper is indicative of the plane of a distal curve. In some variations, orientation indicators may have multiple apices that are aligned with multiple curves in multiple planes, which may help the practitioner position and orient the distal portion of the tissue removal device as desired. The orientation indicator may be attached to the cannula and/or stylet by soldering, welding, adhesive bonding (e.g., 3311 UV adhesive that may be UV cured), snap fit, or other appropriate methods. In some variations, the orientation indicator may be attached or integrally formed with a proximal connector of the cannula and/or stylet. This may provide a mechanism for the cannula and stylet to be coupled together in a particular orientation.

Cannulas and stylets may each have proximal connectors that couple them to each other. The proximal connector of a cannula may also be used to couple it with a tissue removal device, e.g., a collector port and/or travel limiter. Connectors may be any standardized connector (e.g., any luer-type connectors, screw-type connectors, taper ground joints, etc.), or may be a proprietary connector. In some variations, a cannula may have a male-type connector that is configured to connect with a stylet or tissue removal device with a female-type connector. Engagement of the proximal connectors of cannula, stylets, and/or tissue removal devices may prevent relative movement between the devices. In some variations, when a stylet is connected to a cannula, the stylet may not be able to move longitudinally within the cannula, but may be axially rotated within the cannula. This may allow a practitioner to adjust the alignment between the cannula and stylet during the insertion of the cannula and stylet into the body. Alternatively or additionally, engagement of the proximal connectors between a cannula and stylet, or a cannula and a travel limiter of a tissue removal device may prevent relative longitudinal and axial motion between the devices. Locking the orientation and position between the cannula and stylet (and/or cannula and travel limiter) may help prevent inadvertent device misalignment or movement during a procedure. Travel limiters are disclosed, for example, in U.S. patent application Ser. No. 13/332,227, which is incorporated herein by reference in its entirety.

In some examples, the distal region of the cannula and/or stylet may comprise a radio-opaque structure (e.g. rings or bands) to facilitate confirmation of its position using radiographic imaging. In other examples a separate radiographic marker instrument may be used to confirm and evaluate the cannula placement.

In some variations, a bendable flexible curved cannula may be used in association with either a straight stylet or a curved stylet to obtain curved access to a spinal area. A curved access pathway not only offers a larger tissue removal zone at one target site, but it may also provide flexible access to multiple target sites in one or more herniated discs. A curved or non-linear access pathway that may be provided by a bendable flexible curved cannula may be shorter than a straight access pathway, and may be less disruptive to surround tissue structures.

It may also provide better orientation towards the middle of a disc, as compared with a straight access pathway.

The bending range of the curved cannula may be in the range of from about 10 degrees to about 80 degrees, sometimes from about 20 degrees to about 70 degrees, and other times from about 30 degrees to about 60 degrees, and still other times from about 40 degrees to about 50 degrees. The curved distal portion 2914 may comprise a radius of curvature of about 0.5 centimeter to about 30 centimeters; sometimes about 1 centimeter to about 20 centimeters, sometimes about 5 centimeters to about 15 centimeters and other times about 8 centimeters to about 10 centimeters. When the curved distal portion is straightened, the curved cannula may comprise a length of about 4 inches to about 12 inches or more, sometimes about 5 inches to about 10 inches, and other times about 6 inches to about 9 inches.

Prior to inserting the tissue removal device into the cannula, approximately 0.5 cc of saline may be injected into the disc through the cannula. Under image guidance, the tissue removal device may be inserted through the cannula until the target site has been reached. Using image guidance, the practitioner may advance the tip of the tissue removal device to the full plunge depth, and confirm that the tip is in a safe location. The tissue removal device may then be actuated. The placement of the device in the course of tissue removal may be intermittently confirmed by fluoroscopy or another appropriate imaging modality. The tissue removal device may be used until sufficient tissue material has been removed, and/or the collector is full. In some variations, a negative pressure source may be coupled to the collector which may help expedite tissue removal. The markings on the collector indicate the quantity of tissue removed. The tissue removal device may be turned on and used continuously for about 0.5 second to about 6.0 minutes, e.g., 2.0 minutes.

Once a sufficient quantity of tissue material has been removed, the tissue removal device may be turned off. The above steps may be repeated until the desired quantity of tissue has been removed. If additional treatment is required within the disc, the straight or curved stylet may be reinserted into the cannula, and the cannula may be repositioned. In some procedures, it may be desirable to limit the total run-time of the tissue removal device to about 6.0 minutes or less. The straight stylet may be inserted into the cannula and fixedly attached at the proximal hub. Then, the cannula-straight stylet assembly may be withdrawn from the access site. In some variations, the battery of the tissue removal device may be removed and disposed according to local regulations.

The cannula, stylet, and tissue removal devices described above may be used to perform a discectomy. The devices may be used in a minimally invasive procedure, or an open surgery procedure. The cannula-stylet assembly may be used to form a passageway or a working channel through the tissue about a target site in the spinal region. For example, to perform a discectomy procedure, the patient may be prepped and draped in the usual sterile fashion and in a lateral decubitis or prone position. General, regional or local anesthesia may be achieved. A straight stylet with a sharp distal tip may be inserted into the lumen of a straight cannula. The assembly may then be percutaneously inserted through a posterior or posterolateral entry point on the back of the patient. The cannula-stylet assembly may be further inserted into the epidural space or into the paravertebral space, depending on the assembly's point of entry. Alternatively, the assembly may be used to penetrate the disc annulus directly from a point of entry further away from the midline of the patient's back. In some embodiments, the assembly may be introduced on the ipsilateral side from which the nerve impingement has been identified and at an angle of about 25 degrees to about 45 degrees to the patient's back. In other procedures, a contralateral approach and/or a different angle may be used. In alternative embodiments, an anterior procedure through the abdominal cavity of the anterior neck region may be performed.

The cannula-stylet assembly may be advanced together to a target tissue site, as described above. During the insertion of the assembly, the stylet may be independently rotatable such that the operator may adjust the orientation of the optional beveled edge of the stylet in order to form a passageway through the surrounding tissue, bones or other anatomic structures. The insertion of the cannula-stylet assembly may be performed under the guidance of external imaging and/or visualization techniques.

Fluoroscopy and/or CT scan may be used before, during and/or after the procedure to assess the patient's anatomy, the position of the instruments, the structural changes after tissue removal, and/or to verify the integrity of the disc. In some variations, a small amount of radiopaque contrast agent may be injected into the disc space to enhance visualization. Such injection may be performed by the tissue removal device through an infusion or irrigation channel, or through the aspiration port. In other variations, the cannula may comprise an infusion or irrigation lumen to introduce the contrast agents. In some variations, the tissue removing procedure may be assessed by the quantity and/or color of the tissue removed through an optically transparent chamber, or collection chamber. Upon completion of the procedure, the tissue removal device may be proximally withdrawn, followed by withdrawal of the cannula.

Devices described herein may be used with one or more visualization systems, such as one or more endoscopic visualization systems, as appropriate.

It is to be understood that this invention is not limited to particular exemplary variations described, as such may, of course, vary. It is also to be understood that the terminology used herein is for the purpose of describing particular variations only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a blade” includes a plurality of such blades and reference to “the energy source” includes reference to one or more sources of energy and equivalents thereof known to those skilled in the art, and so forth.

The publications discussed herein are provided solely for their disclosure. Nothing herein is to be construed as an admission that the present invention is not entitled to antedate such publication by virtue of prior invention. Further, the dates of publication provided, if any, may be different from the actual publication dates, which may need to be independently confirmed.

What is claimed is:

1. A device, comprising:
a handle; and
a tissue removal mechanism coupled to the handle, the tissue removal mechanism including:
a tubular member having a lumen therethrough terminating in a distal opening;
an elongate member rotatably and slidably disposed within the lumen of the tubular member, a proximal end of the elongate member coupled to a drive source to impart rotational movement thereof, a distal end of the elongate member including an impeller, the elongate member being configured to advance from the distal opening of the tubular member, such that a portion of the impeller is distal to the distal end of the tubular member.

2. The device of claim 1, wherein the impeller includes a blunt distal tip.

3. The device of claim 1, wherein the tissue removal mechanism further comprises a tissue collection chamber coupled to the lumen of the tubular member.

4. The device of claim 1, wherein the tissue removal mechanism further includes a helical member disposed about a portion of the elongate member.

5. The device of claim 1, wherein the tubular member includes a distal tip including an opening.

6. The device of claim 5, wherein the opening is formed by a protrusion.

7. The device of claim 6, wherein the protrusion is a first of a plurality of protrusions, the plurality of protrusions being configured to form a serrated edge.

8. The device of claim 5, wherein the opening includes an edge, the impeller being configured to cooperate with the edge to facilitate tissue removal.

9. The device of claim 8, wherein the edge is a sharpened edge.

10. The device of claim 1, wherein the impeller includes a helical flute.

11. The device of claim 10, wherein the helical flute of the impeller is configured to cooperate with the lumen to break a target tissue into a plurality of target tissues.

12. The device of claim 10, wherein the helical flute of the impeller is configured to cooperate with the distal opening of the tubular member to facilitate tissue removal when a distal tip of the impeller is advanced out the distal opening of the tubular member.

13. The device of claim 1, wherein the elongate member includes a distal end, the distal end including an angled surface.

14. The device of claim 1, wherein the drive source is an electric motor.

15. The device of claim 1, wherein the elongate member comprises the impeller and a tubular member having a distal end coupled to the impeller and a proximal end coupled to the drive source.

16. The device of claim 1, further including a power source configured to provide power to the drive source, the power source being located within the handle of the tissue removal device.

17. A method, comprising:
advancing the device of claim 1 to a target tissue;
advancing the elongate member such that a portion of the impeller is distal to the distal opening of the tubular member; and
removing a portion of the target tissue with the tissue removal mechanism while the portion of the impeller is distal to the distal opening of the tubular member.

18. The method of claim 17, wherein removing the portion of the target tissue with the tissue removal mechanism includes removing a portion of target tissue with the portion of the impeller that is distal to the distal opening of the tubular member.

19. The method of claim 17, wherein the tissue removal mechanism further comprises a tissue collection chamber, the method including gathering the portion of the target tissue in the tissue collection chamber.

20. The method of claim 17, wherein the portion of the target tissue is a first portion and the opening of the tubular member is a first opening, the tubular member including a second opening, the method including removing a second portion of the target tissue through the second opening of the tissue removal mechanism.
